(19) United States
(12) Patent Application Publication
(10) Pub. No.: US 2007/0231850 A1
(43) Pub. Date: Oct. 4, 2007

(54) Patterned Cell Network Substrate Interface and Methods and Uses Thereof

(76) Inventors: Mealing Geoffrey, Ottawa (CA); Christophe Py, Ottawa (CA); Denhoff Mike, Ottawa (CA); Reza Dowlatshahi, Kanata (CA); Karim Faid, Nepean (CA); Raluca Voicu, Gatineau (CA); Mahmud Bani, Ottawa (CA)

Correspondence Address: NATIONAL RESEARCH COUNCIL OF CANADA;1200 MONTREAL ROAD, BLDG M-58, ROOM EG12, OTTAWA, ONTARIO, K1A 0R6, CA

(21) Appl. No.: 11/579,729

(22) PCT Filed: May 5, 2005
(86) PCT No.: PCT/CA05/00682

§ 371 Date: Nov. 6, 2006

Related U.S. Application Data
(60) Provisional application No. 60/568,220, filed on May 6, 2004.

(51) Int. Cl. C12M 1/14; C12M 1/34; C12Q 1/02

(52) U.S. Cl. 435/29; 435/288.4

(57) ABSTRACT

There is provided herein a method and apparatus suitable for use in studying cell membrane related activities. Activities of interest include patch-clamp related studies of networks of cells on a solid substrate. Cells are grown, preferably in a patterned manner, on a substrate having microholes therein. Seals between the cells and the microholes are formed. Each microhole is attached to a channel. In many cases only one hole will be attached to a single channel, allowing examination of effects of a stimulus at a number of different points in a network of one or more cell types. This may be interest, for example, to those wishing to study interactions between neurons or neuromuscular junctions.

### FIELD OF THE INVENTION

The invention relates to methods and materials suitable for use in growing and monitoring two-dimensional networks of living cells on a substrate.

### BACKGROUND TO THE INVENTION

A number of methods for studying cell-to-cell communication are known, including: conventional patch-clamp techniques (glass micropipette coupled to peripheral electronics); sharp electrode intracellular recording; field potential recordings; and using ion or voltage-sensitive fluorescent dyes.

Conventional patch-clamp, or sharp electrode recording techniques are generally too slow and difficult to interrogate multiple interconnected cells, making them inadequate for studying communication in complex engineered cell networks, or for screening potentially therapeutic pharmaceutical compounds for efficacy at intended ion channel targets or liability at unintended targets. Voltage, or ion-specific fluorescent dyes give an indirect measure of ion channel function and may lack the speed required to resolve fast physiological events.

US 2004/0251145 A1 of Robertson (published 16 Dec. 2004) depicts an apparatus for use in monitoring properties of ion channels in cells. Essentially, this application is understood to teach the formation of blind pools under a portion of cell membrane. An electrode extends into the pool and is sealed to prevent leakage along the electrode's path. Such an apparatus is unsuitable for studying the effect of localized delivery of agents and for the study of whole-cell effects.

### SUMMARY OF THE INVENTION

In an embodiment of the invention there is provided a method of studying cell membrane related activities, said method comprising:

(a) obtaining a cell adhesion surface having discrete orifices therein in communication with attached channels;

(b) culturing cells on the cell adhesion surface so that at least some of the cells grow over at least one orifice, such that the portion of the cell membrane in contact with the outer perimeter of the orifice forms a seal with the cell adhesion surface in the area immediately surrounding the orifice; and

(c) measuring changes in conditions within a fluid located within the channels connected to the orifices.

In an embodiment of the invention there is provided a substrate. The substrate comprises: a microhole containing layer having microholes extending through it; a guidance layer of substantially inert material sealably engaging portions of a first side of the microhole containing layer; said guidance layer in combination with the microhole containing layer defining a series of troughs extending substantially parallel to the microhole containing layer surface, wherein the trough walls are formed at least in part by the guidance layer and the trough base is defined at least in part by a region of the microhole containing layer defining a microhole.

In an embodiment of the invention there is provided a method of producing a substrate suitable for use in attaching and/or growing cells so as to promote development of structured cell networks in two or more dimensions. The method comprises: a) obtaining a film on a first side of a substantially inert backing; b) creating microholes in the film; c) bonding the second side of the backing to a carrier; d) obtaining a mask in the first side of the backing and creating windows in the thin film mask, said windows being aligned so as to connect to a microhole; f) etching the backing through the windows in the mask, to create an inverted pyramid structure resulting in a membrane including the micro-hole; g) obtaining a second chip defining channels; h) bonding the second chip to the backing such that a channel is positioned over a microhole in substantially sealing engagement; i) releasing the backing from the carrier; j) applying a patterned growth cell guidance region on the first side of the membrane in alignment with micro-holes such that a micro-hole is located at the bottom of a well and the well is connected to other wells via trenches; k) coating the resulting product with a bio-compatible, electrically insulating plastic so as not to plug the micro-hole, and polylysine or another suitable thin-film to promote the implantation of different types of cells.

In an embodiment of the invention there is provided a method of producing a substrate suitable for use in growing cells so as to promote growth of structured networks in two or more dimensions. The method comprises: a) obtaining a film on a first side of a Si wafer with a crystalline orientation; b) creating microholes in the SiN/Au thin film; c) bonding the second side of the wafer to a carrier with wax or another sacrificial layer; d) obtaining a mask in the back of the wafer and creating windows in the thin film mask, said windows being aligned so as to connect to a microhole; f) etching the Si wafer through the windows in the mask, thereby creating an inverted pyramid structure resulting in a membrane including the micro-hole; g) obtaining a second chip defining channels with a defined pitch; h) bonding the second chip to the Si chip such that a channel is positioned over a microhole in substantially sealing engagement; i) releasing the Si chip from the carrier; j) defining a network of wells and trenches in alignment with micro-holes such that a plurality of micro-holes are located at the bottom of a well and connected to other wells via trenches; k) coating the entire chip with a bio-compatible material so as not to plug the micro-hole, and polylysine or another suitable thin-film to promote the attachment, growth and/or guidance of different types of cells.

In an embodiment of the invention there is provided a method of producing a substrate suitable for use in growing cells so as to promote growth of structured networks in two or more dimensions. The method comprises: a) obtaining a tip connected to a beam; b) obtaining a backing having a carrier bonded to a first surface, said backing defining towers and walls along a second surface; c) positioning the tip such that apex of the tip in contact with the top of a tower on the backing and the beam extends to and edge of the backing; d) filing the space between the tip and the backing with a material which is fluid when applied but can be converted to a solid form; e) converting the material of step d into a solid form; f) removing the tip and the backing from the cured material to reveal a well structure with microholes and channels therein; g) where the tip was positioned such that its removal results in openings to the outside air in regions formed by the tip or the beam, closing off such openings to form closed channels except at the end of the channels defined by the beam; h) optionally, coating the resulting product with a bio-compatible, electrically insulating plastic so as not to plug the micro-hole, and polylysine or another suitable thin-film to promote the implantation of different types of cells.

In an embodiment of the invention there is provided a method of forming an interface between a biological membrane and a substrate. The method comprises: a) obtaining a substrate of claim 5; b) culturing cells on the microhole containing layer/guidance layer surface of the substrate; c) creating a patch-clamp connection between the cell and the substrate at a microhole.

In an embodiment of the invention there is provided a method of producing a system suitable for use in studying whole-cell electrical responses to a stimulus. The method comprises: obtaining a substrate as described herein; culturing cells on the membrane/guidance layer surface of the substrate in a culture medium such that at least one cell grows over a microhole; creating a patch-clamp connection between the membrane and the substrate at a microhole; and, rupturing a portion of the membrane over the microhole.

### BRIEF DESCRIPTION OF THE DRAWINGS

FIG. 1 is schematic representation of an embodiment of a patterned cell network substrate interface and a use thereof in patch clamp investigations.

FIG. 2 is a schematic representation of an embodiment of an approach to the fabrication of a substrate for use as described in FIG. 1.

FIG. 3 depicts scanning electron micrograph pictures of embodiments of fluorinated PDMS stamps with (a) channels and (b) pillars used as secondary moulds and (c) and (d) their respective replicated PDMS microstructured substrates.

FIG. 4 is a depiction of cells, microstructures and surface chemistry modifications relating to Example 1.

FIG. 5 is a further depiction of cells, microstructures and surface chemistry modifications relating to Example 1.

FIG. 6 is a depiction of results of Example 1 part C showing excitable neurons functionally connected as assessed by calcium imaging and electrophysiology.

FIG. 7 is a schematic depiction of an embodiment of a substrate for a patterned cell network.

FIG. 8 depicts (A) a schematic representation of an embodiment of a substrate, and (B) an embodiment of a Si-based substrate fabrication process.

FIG. 9 is a series of depictions of embodiments the substrate.

FIG. 10 is a depiction of an embodiment of a PDMS based fabrication approach in a sectional view.

FIG. 11 is a depiction of an embodiment of a PDMS based fabrication approach in a plan view.

FIG. 12 is a depiction of a prior art AFM tip.

FIG. 13 is a depiction of alternative tip configurations useful in certain embodiments of the invention.

FIG. 14 is a depiction of an embodiment of the process for fabrication of the substrate using a tip.

### DETAILED DESCRIPTION OF THE PREFERRED EMBODIMENTS

The present invention provides, in one aspect, an apparatus and method to grow cells, including neurons, on substrates with patterned guidance pathways printed on their surface such that they form structured 2-D networks. In an embodiment of the invention the apparatus and method permits study of cell interfaces and/or a means to interrogate cell function and intercellular communication. The structured 2-D cell networks may be interfaced with a patch-clamp platform that allow simultaneous recording, or stimulation of individual cultured cells in the network. This is accomplished by using an alternative to recent “patch-on-chip” technology that has been applied to isolated cells in suspension, but which is not suitable for use with cells growing on a substrate.

Traditional/conventional patch pipettes are constructed from various types of glass (see Hamill et al., 1981). Formation of a very high resistance electrical seal (in the order of gigaohms) between the pipette (or the planar chip) and the cell membrane is widely considered essential to permitting the measurement of small ionic currents indicative of ion channel function. Unfortunately, the molecular nature of this “gigaseal” is still not well understood (see Corey & Stevens, 1983; Opsahl & Webb, 1994). “Patch-on-chip” technology essentially replaces the patch-clamp pipette with a planar array of micron-size apertures, in the surface of a glass (e.g. Fertig et al., 2002) or silicon polymer substrate (e.g. Klemic et al., 2002). Gigaseal formation is accomplished by, either physically positioning the patch pipette in very close proximity to the cell, or by positioning the suspended cell over an aperture in the planar chip, and then applying suction to draw the cell membrane to the pipette or chip substrate such that molecular forces are exerted over a nanometer distance. Note that, with both technologies, physical alignment and suction are required for gigaseal formation.

Planar patch-clamp technology has not been applied to cells grown in culture, in organized networks. This would be tremendously useful, since ion channel activity or membrane potential could be monitored simultaneously in multiple cells connected in a well-defined circuit for extended durations. For example, in the case of neurons, pre- and post-synaptic events can be monitored. In hybrid substrate systems, events at the neuromuscular junction can be monitored. Similarly, interactions between neurons and glia can be evaluated. This represents a sophisticated alternative or addition to pharmacological screening using isolated cells in suspension.

Cues, such as surface chemistry (Castner & Ratner, 2002), topography (Wilkinson et al., 2002; Mahoney et al., 2005) and mechanical properties (Wong et al., 2004) can be manipulated to influence cell growth on a substrate. Patterned microstructures have been used as tools to position cells (Ratner & Bryant, 2004), and alterations to nano-scale surface topography, made by etching silicon wafers, have been used to guide growth through interactions with growth cone filopodia (Fan et al., 2002). Microcontact printing methods, such as lithographically applied polylysine-conjugated laminin patterns, have also been used to guide neuron attachment and axonal outgrowth (James et al., 2000).

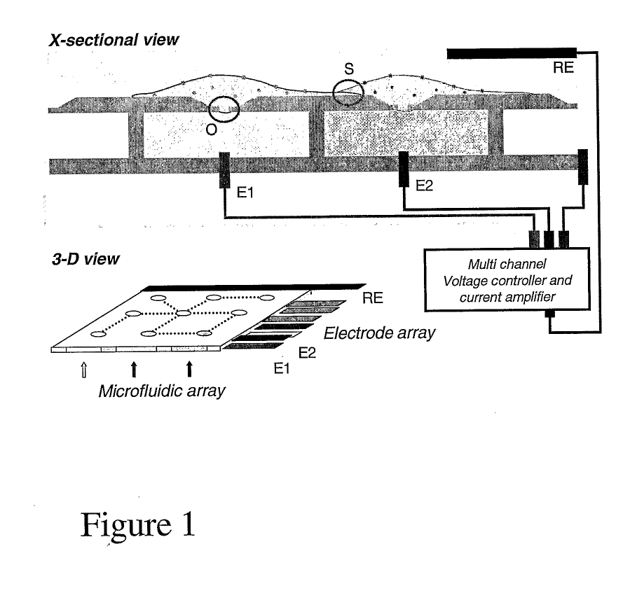

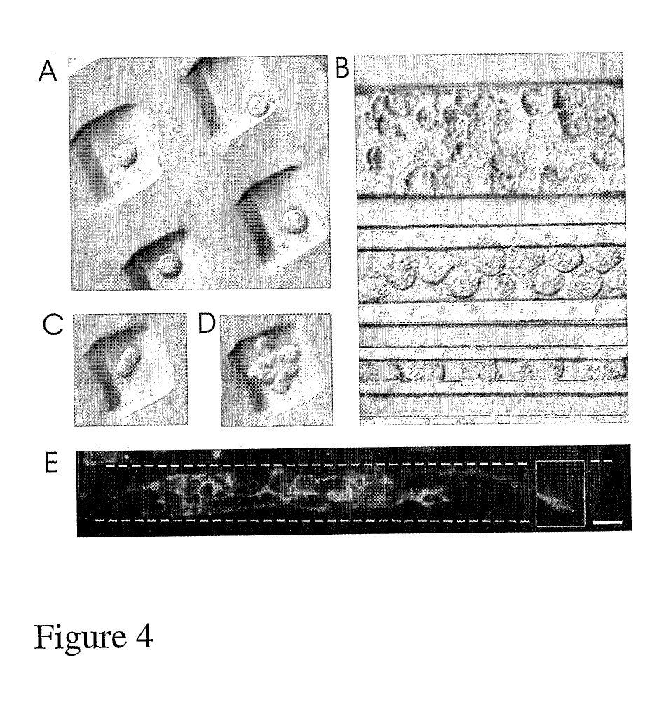

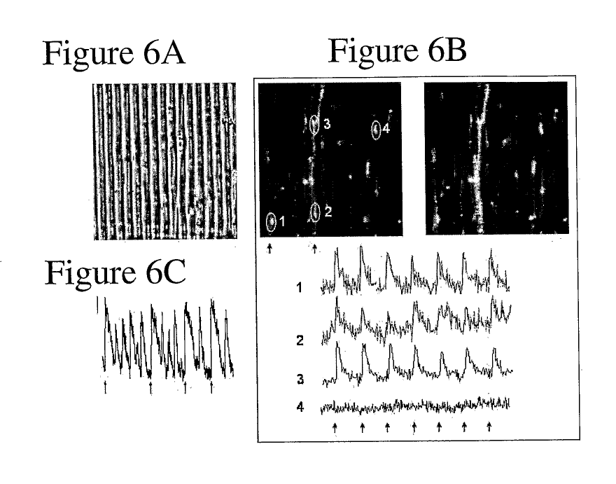

FIG. 1 depicts a schematic drawing of novel planar patch-clamp interface for neurons in a synthetic network grown on a patterned substrate. Neurons are positioned using locating wells (similar to those shown in FIG. 4a) over 2-4 μm diameter orifices (O) which individually communicate with a specific subterranean fluidic channel housing an electrode. A high resistance seal between the cell membrane and the perimeter of the orifice ensures detection of current flow through ion channels in the membrane patch covering the orifice. Neurite growth is directed towards neighbours using guidance pathways (dotted lines), similar to the patterned channels shown in FIG. 6a. Electrodes in the subterranean microfluidic array are connected to a multichannel voltage controller/current amplifier and referenced to an electrode in the upper perfusion chamber.

The above cues can be used to manipulate cell/extracellular matrix/substrate interactions and guide cell positioning and attachment in the substrate and subsequent growth and connectivity in culture. By aligning and growing cells on a surface with small apertures that connect to a “subterranean structure, a high resistance (gigaohm) electrical seal that “partitions off” a small circular area (e.g. 0.5-10 μm diameter) of the membrane surface (area in left side circle in FIG. 1) is achieved. The apertures are connected to individual microfluidic perfusion channels and electrodes. This allows the recording of currents resulting from ion channel activity in the region of membrane over the orifice (essentially cell-attached patch-clamp). In some instances, a pore-forming antibiotic may be perfused into the area beneath the orifice to create a “perforated patch”, permitting the recording of “whole-cell” currents resulting from ion channel activity through the entire cell membrane. The subterranean architecture is constructed such that each orifice (and each cell) is connected to a different microfluidic channel and electrode. This permits the study of multiple cell-cell communications through gap junctions, or synapses (area shown in right side circle in FIG. 1).
In some instances it will be desired to select an embodiment of the invention germinating creation of a planar patch-clamp interface suitable for use with cells grown in culture and especially those grown in organized patterns.
In some instances it will be desired to select an embodiment of the invention allowing specific manipulation of the intracellular or extracellular environment in individual cells that are connected together in a network.
In some instances it will be desired to select an embodiment of the invention allowing use of the interface as a novel tool to study synaptic function in organized networks of neurons, or to study cell-cell interactions in other cell types.
In some instances it will be desired to select an embodiment of the invention allowing use of the interface as a novel high-throughput electophysiological drug screen using cultured cells grown in organized patterns.
Having the ability to genetically manipulate individual cells and examine their effects on neighbouring cells
Creating homogeneous and heterogeneous neural networks by differentiating adjacent neural progenitors into different neural subtypes

It will be appreciated that different configurations and dimensions of the apparatus of the invention are specifically contemplated. By way of non-limiting example, the surface for cell surface attachment, the size of the orifices, the perfusion channel dimensions and lengths and the microfluidic perfusion system may be selected to optimize use of the apparatus for particular cell types, for particular growth conditions, and/or to study particular phenomena.

In some instances, the surface for cell attachment will be poly-dimethylsiloxane (PDMS) or any other suitable silicone derivative, laminin, collagen, structural extracellular matrix proteins, nanopatterned surfaces, proteins that modulate cellular interaction with the extracellular matrix, and/or polylysine.

In some instances, the orifice size will be between about 1 to 10 μm. In some instances 2-8, in some instances 3-7 μm.

In some instances, the orifice may be fitted with a sieve structure to provide support to the cell membrane, while still allowing membrane perforation.

While the apparatus is described primarily with reference to the example of mammalian cells or mammalian-derived cells as the cells for examination, it will be appreciated that the invention includes embodiments useful in the study of other cell types, including bacterial cells. Dimensions and coating of the apparatus is preferably adjusted in light of the known preferences and size of the cells to be examined. For example, to study signalling within a bacterial colony, smaller orifices would be employed and an appropriate substrate coating such as polystyrene or agar would be employed. In light of the disclosure herein, one skilled in the art of the bacteria in question could readily select appropriate parameters.

The medium or other fluid employed within the perfusion channels will be selected based on known features of the medium or other fluid, the cells to be examined, and the nature of the investigation.

In some instances, exogenous signalling messengers or other materials to induce or trigger a cellular response may be included in or added to the medium or other fluid to allow examination of the resulting cellular response or to manipulate (genetically or otherwise) cell development.

Ion- or voltage-sensitive dyes will in some instances be introduced in the chip perfusion channels and changes (eg. Fluorescence) in the dyes are monitored in parallel to electrical activity resulting from ion channel activity.

There is provided in an embodiment of the invention an apparatus useful in the investigation of cell-to-cell communication between cells of interest said apparatus comprising:
a cell adhesion surface adapted to permit the attachment and growth of cells of interest;
the surface defining a plurality of discrete orifices;
channels each having a cell end and a fluidic circuit bypassing the orifice;
the cell end of each channel in sealed connection with a single orifice;
the fluidic circuit being adapted to permit operative connection of an electrode so as to permit the taking of measurements within the channel, as well as the circulation of fluids without going through the orifice.

In some instances it may be desirable to obtain multiplexed or combined readings arising from cell activities. This can be accomplished by compiling electrical signals from electrodes located in different channels.

### EXAMPLE 1

1) Cell Placement and Directed Growth: Example of Realisation

Hybrid silicon-polymer chips with microscale topography and contrasting surface chemistries were created using a novel combination of soft lithography techniques, and evaluated for their suitability as a platform to guide cell attachment, growth and differentiation. These capabilities are all desirable to synthesize organized neural networks in vitro. Neurons developed on these chips exhibit patterned growth and functional communication, evidenced by spontaneous and stimulated action potentials and intracellular calcium oscillations. Integration of patch-clamp technology into this platform to create a novel long-term interface with the formation of a high resistance (giga-ohm) electrical seal between the cultured cell membrane and the perimeter of a micron-sized orifice integrated into the substrate in light of the disclosure herein has potential as a tool to investigate mechanisms underlying neurogenesis, synaptic transmission, and neurodegeneration. It may also lead to the development of more sophisticated and functionally relevant bioassays and high throughput electrophysiological screening, thus speeding the drug discovery process.

A. Microstructure Fabrication and Surface Chemistry Modification

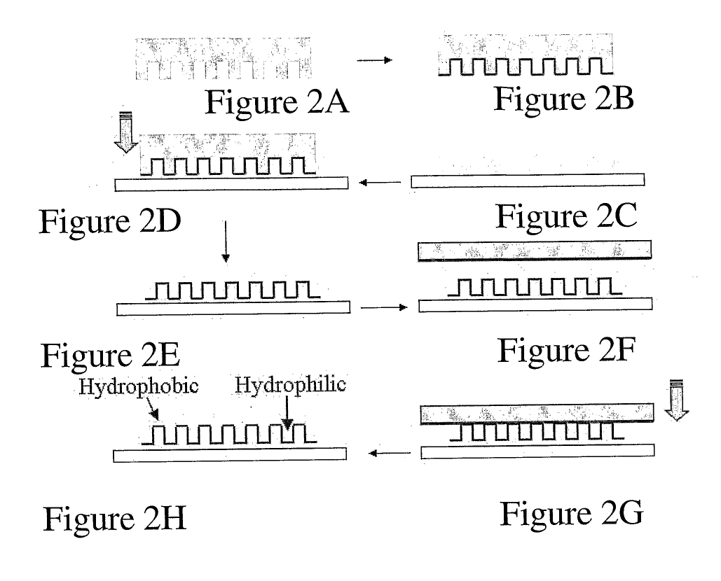

The general protocol used for the rapid and efficient fabrication of the polymer microstructures and their subsequent chemical patterning is outlined in FIG. 2.

FIG. 2 depicts the fabrication and chemical patterning of PDMS microstructures. a) PDMS stamp with 10 μm deep channels hydrophilized by air plasma to introduce silanol groups on the surface. b) Chemisorption of a fluoro-siloxane derivative on the surface and curing in aqueous solution to form a highly hydrophobic surface. c) Spin coating of thin layers of uncured PDMS precursor on glass substrate. d) Imprinting of the microstructures by the fluorinated stamp and curing by heating. e) Hydrophilic microstructures created by air plasma. f) Chemical patterning of the PDMS microstructures through the introduction of hydrophobic functional groups. g) Transfer of the fluorinated siloxane and curing. h) PDMS microstructures with a dual hydrophobic-hydrophilic character.

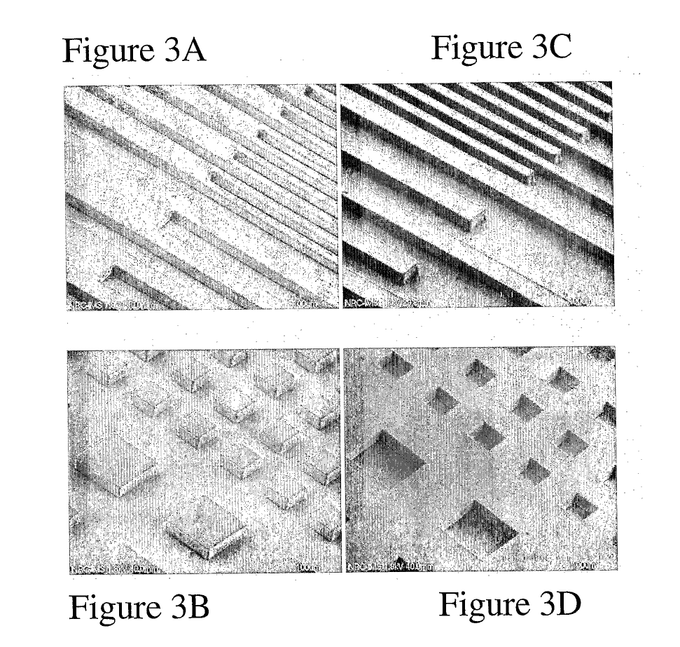

A flexible stamp was made by replicating PDMS, using Sylgard 184 kit and a silicon wafer patterned with an SU8 negative photoresist (Microchem), having microsized features as a master mold following published procedures (Xia, 1998; Bensebaa, 2004). The microstructures on the master mold consist of, either 5, 10, 25, 50 and 100 μm wide recessed lines spaced by the same width or of square pillars of 5, 10, 25, 50 and 100 μm spaced by the same dimensions. The thickness of the SU8 photoresist was set to 10 μm. The replicated PDMS stamp (FIGS. 3a and 3b) exhibits features that are complementary to those of the master SU8-silicon mold. The PDMS stamp was subsequently washed in a Soxhlet setup using ethanol for 3 hours to remove any unreacted oligomers. The washed PDMS stamp was characterized by contact angle (112.7°), XPS and ATR-FTIR.

The PDMS stamp was rendered hydrophilic by creating —OH groups on the surface in an air plasma reactor for 1 minute at 2×10⁻¹ mbar. The PDMS-OH stamp shows a very low contact angle (6.4°) and was stored in de-ionized water prior to further modification in order to preserve its hydrophilic character. The patterned PDMS-OH substrate was immersed overnight in 100 mM heptadecafluoro-1,2,2,2-tetrahydrodecyl-triethoxysilane (HFS) solution in ethanol. The fluorosilane-modified patterned PDMS (PDMS-CF3) (FIG. 2b) was rinsed thoroughly with ethanol after incubation and immersed in H₂O for polycondensation of the siloxane groups, yielding highly hydrophobic substrates with a contact angle of 112.8°. XPS and ATR-FTIR studies are in agreement with those reported in the literature for similar materials and indicate that the fluoro-silane derivative is covalently attached to the surface of the secondary PDMS mold.

This non-sticking PDMS stamp was put in conformal contact (FIG. 2c-d) with an uncured polymer film spin-coated on a glass cover slip (a polystyrene Petri-dish or a silicon wafer also worked) and cured thermally. A film of uncured PDMS prepolymer was spin-coated at 2000 rpm to yield a thickness of 25-30 μm, imprinted by the secondary PDMS mold and cured at 90° C. for 2 h. After curing, the fluorinated stamp was easily removed from the substrate. The features transferred to the polymer substrate were found to be complementary and virtually identical to those of the stamp (FIGS. 3c and d). The imprinted samples were characterized by optical microscopy, scanning electron microscopy, and profilometry. It was found that the fluorinated stamp could be used multiple times, without mechanical or chemical degradation. The master mold, made by a standard lithography technique, could also be re-used.

The imprinted PDMS was rendered hydrophilic in an air plasma reactor. The imprinted PDMS-OH was stored in de-ionized water prior to further modification in order to preserve its hydrophilic character. A flat PDMS, obtained by thermal polymerization of a PDMS prepolymer in a polystyrene Petri-dish, was inked with HFS for 30 minutes, dried with nitrogen and then put in conformal contact with the top of the imprinted PDMS-OH for 90 minutes using a modification of a published methodology (Pfohl, 2001). After removing the stamp, the imprinted PDMS now had a dual character: hydrophobic (fluorinated) on top of the channels and hydrophilic (silanols) inside the wells or the bottom of the channels. The samples were stored in water insuring the polycondensation of the siloxane groups on top of the imprinted substrate and the conservation of the silanol groups in the bottom and the walls. Micro-XPS imaging indicated that only the tops of the imprinted microstructures contain fluorine (see FIG. 2). A flexible stamp was made by replicating PDMS, over a master mold following published procedures (Bensebaa et al., 2004). The microstructures on the master mold consisted of either 5, 10, 25, 50 or 100 μm wide recessed lines spaced by the same width or square pillars of 5, 10, 25, 50 or 100 μM spaced by the same dimensions. The replicated PDMS stamp (FIGS. 3a,b) exhibited features complementary to those of the master SU8-silicon mold. The PDMS stamp was rendered hydrophilic by creating —OH groups on the surface in an air plasma reactor for 1 min at 2×10⁻¹ mbar. The patterned PDMS-OH substrate was immersed overnight in 100 mM heptadeca-fluoro-1,2,2,2-tetrahydro-decyl-triethoxysilane (HFS) solution in ethanol. The fluorosilane-modified patterned PDMS (PDMS-C₃F) (FIG. 2b) was rinsed with ethanol after incubation and immersed in H₂O for polycondensation of the siloxane groups, and yielded highly hydrophobic substrates with a contact angle of 112.8°, indicating that the fluoro-silane derivative was covalently attached to the surface of the secondary PDMS mold.

The PDMS stamp was put in conformal contact (FIGS. 2c,d) with an uncured PDMS film spin-coated at 2000 rpm to a thickness of 25-30 μm on a glass coverslip, and thermally cured at 90° C. for 2 h. After curing, the two PDMS stamps were substantially effortlessly separated due to fluorination of the first stamp. The features transferred to the polymer substrate were complementary and virtually identical to those of the first stamp (FIGS. 3c,d). The imprinted PDMS was rendered hydrophilic in an air plasma reactor as shown. The imprinted PDMS-OH was stored in de-ionized water prior to further modification in order to preserve its hydrophilic character. A flat PDMS surface, obtained by thermal polymerization of a PDMS prepolymer in a polystyrene Petri-dish, was inked with HFS for 30 minutes, dried with nitrogen, and then put in conformal contact with the top of the imprinted PDMS-OH for 90 min (FIGS. 2f,g) using a modified methodology (Li et al., 2001).

After removing the stamp, the imprinted PDMS now had a dual character: hydrophobic (fluorinated) on the upper surface and hydrophilic (silanols) inside the wells or channels (FIG. 2h).

B. Patterned PDMS Substrates Provide a Scaffold for Cell Positioning, Guidance, and Proliferation

The efficacy of these fabricated substrates as a platform to create simple neural networks was evaluated. N2a neuroblasts (ATCC, Manassas, Va.) were cultured on the test substrates. Similarly, cortical neurons from embryonic day 13 or 17 mice or embryonic day 17 rats were isolated and plated accordingly.

FIG. 4 depicts microstructures and surface chemistry modifications effectively position N2a cells and guide proliferation. a) Hoffman contrast image showing that 50 μm square hydrophilic wells locate N2a cells and promote rapid attachment. b) Cells undergo division within 10 h and c) a colony has formed within 48 h. d) Similarly, N2a cells position and proliferate in hydrophilic channels 50, 25, and 10 μm wide (top to bottom). Channels narrower than the cell diameter alter cell shape and attenuate proliferation after a few divisions. e) F-actin immunostaining shows N2a cells extend processes along the edge of a 25 μm channel as they differentiate. Inset: deconvolved image of a growth cone guiding the neurite within the channel. Dashed lines represent the boundary of the channel.

Topographic features of the substrate effectively positioned N2a neuroblasts in squares or channels, (FIGS. 4a,d) and the hydrophilic nature of these microstructures promoted selective cell attachment after plating within the boundary of the microstructure. A minimum ratio of 1.5:1 (channel width:cell diameter) was required for N2a cells to proliferate. Cells seeded in 10 μm channels displayed attenuated proliferation and oval morphology. Hence, varying channel width is a potentially useful tool to differentially control growth in a synthetic neuronal network. Histology using F-actin antibody showed neurites guided by their growth cone within the confines of the channel (FIG. 4e).

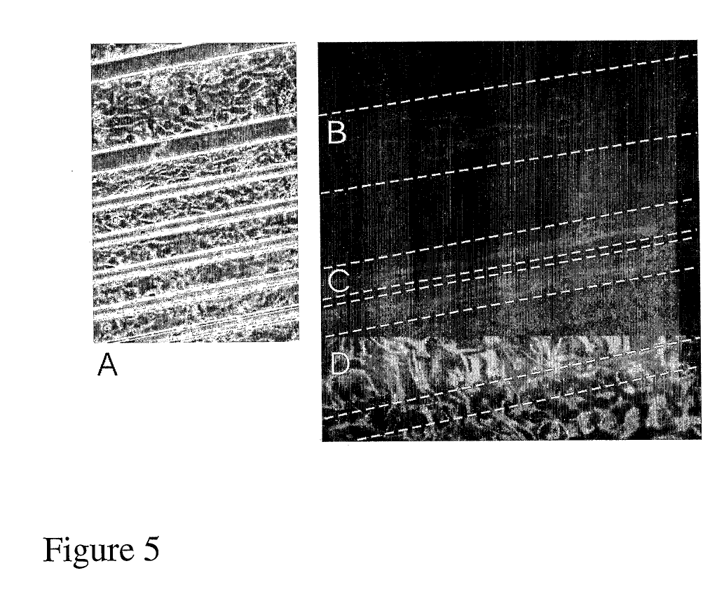

Neural progenitors from E13 mouse cortex were also cultured on microchannel-patterned PDMS substrates. Neurons developed within the confines of the hydrophilic channels and displayed organized parallel architecture similar to that seen in brain substructures (FIG. 5). Unlike N2a cells, they were not hindered in the 10 μm wide channels (FIG. 5c). Remarkably, in contrast to neurons, astrocytes were not influenced by topological or chemical patterning features and grew randomly (FIG. 5d).

FIG. 5 depicts microchannels and surface chemistry modifications effectively position cultured E13 neurons and guide growth. a) Hoffman contrast image showing neurons grown on 50, 25, and 10 μm wide hydrophilic channels (top to bottom). b) MAP-2 staining showing neurons (red) growing in 50 μm, c) or 25 μm wide channels. Dashed lines represent the boundary of the channel. d) GFAP-positive astrocytes are not guided on the same patterned substrate.

C. Assessment of Functionality of Neurons Grown on Patterned Substrates

The excitability and connectivity of cells grown on PDMS substrates with 25 μm wide hydrophilic trenches was examined using N2a cells and E17 cortical rat neurons (FIG. 6a). Intracellular calcium was monitored using Fluo-3 and Fura-red in combination with ratiometric fluorimetry (FIG. 6b). Brief trains (3 s, 10 Hz) of current were applied at 30 s intervals using a bipolar tungsten electrode placed at one end of a PDMS groove. This stimulation paradigm was designed to induce multiple action potentials, sufficient to produce large, prolonged calcium oscillations, detectable with low-speed imaging. Calcium oscillations were recorded in E17 cortical neurons cells propagating along the grooves, demonstrating excitability and functional connectivity. Representative results from 1 of 3 experiments are shown in FIG. 6b. Cells in adjacent channels, distal to the stimulating electrode, were unresponsive, indicating directional propagation of the signal. In similar experiments, N2a cells were unresponsive (n=5).

FIG. 6 depicts simple synthetic neural networks display excitability and connectivity on patterned PDMS substrates. a) Phase contrast image of a PDMS substrate, showing 25 μm wide hydrophylic channels to guide neural growth. Oriented E17 neurons can be observed in these channels. b) Fluorescence images taken 20 s apart show neurons loaded with calcium-sensitive dyes. A stimulating electrode was positioned at one end of the PDMS channels (arrows). Traces 1-4 show relative changes in intracellular calcium concentration at numbered regions of interest identified in the top left image. Stimulation was applied at 30 s intervals (indicated by arrows below traces) to induce calcium oscillations. c) Voltage oscillations recorded from an E17 neuron, using whole-cell patch-clamp. Spontaneous, cyclical waves, composed of multiple action potentials, were observed. It was possible to briefly synchronize this activity using current stimulation (indicated by arrows) through the patch pipette. Scale bar: 10 mV, 1 s.

Membrane potential oscillations were recorded in E17 cortical neurons using whole-cell patch-clamp. Spontaneous, cyclical waves of membrane depolarization, composed of multiple action potentials, were observed, suggesting hyperexcitability, possibly due to a high degree of connectivity in a region at the periphery of the microchannels. Given this hyperexcitability, it was not possible to accurately measure membrane resting potential, but the most polarized potentials recorded were between −43 and −58 mV. It was possible to briefly synchronize this activity using current stimulation, delivered through the patch pipette. Representative results from 1 of 4 experiments are shown in FIG. 6c. In similar experiments, using N2a cells, the membrane resting potential was −23±3 mV and the neuroblastoma cells were unresponsive to stimulation (n=4).

D. Patch-On-Chip Interface with Cultured Neurons in a Synthetic Neural Network

Planar patch-clamp technology has not previously been applied to cells grown randomly in culture, much less to synthetic networks of neurons. In order to allow this an integrated patch-clamp interface was designed to monitor ion channel activity in neurons synaptically connected in a patterned network, which is schematically represented in FIG. 1. This platform permits the extended and non-invasive recording of single ion channel activity in the “cell-attached configuration”. Rupturing the membrane spanning the orifice, using a mechanical or voltage pulse, or by microfluidic application of a pore-forming antibiotic, permits whole-cell recording of populations of ion channels. In addition, a host of fluorescent probes can be introduced into the cytoplasm and monitored simultaneously, using integrated fibre optic sensing.

This apparatus and method enables researchers to simultaneously monitor ion channel function in multiple, synaptically-connected cells in a well-defined circuit for extended durations. This provides a powerful research tool to investigate synaptic function and network signaling. Furthermore, from a pharmacological screening perspective, it presents an attractive alternative to fluorescence intensity plate reader assays, or to electrophysiological assays using isolated cells in suspension.

Biochip Fabrication Methods

In an embodiment of the invention there is provided a method of studying cell membrane related activities comprising:

(a) obtaining a cell adhesion surface having discrete orifices therein with attached channels;

(b) culturing cells on the cell adhesion surface so that at least some of the cells grow over one orifice per cell, such that the portion of the cell membrane in contact with the outer perimeter of the orifice forms a high resistance electrical seal with the cell adhesion surface in the area immediately surrounding the orifice; and

(c) measuring changes in conditions within a fluid located within channels connected to the orifices.

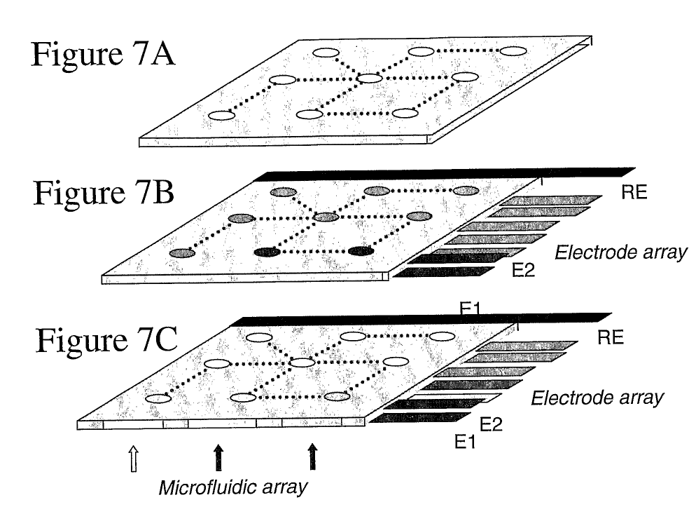

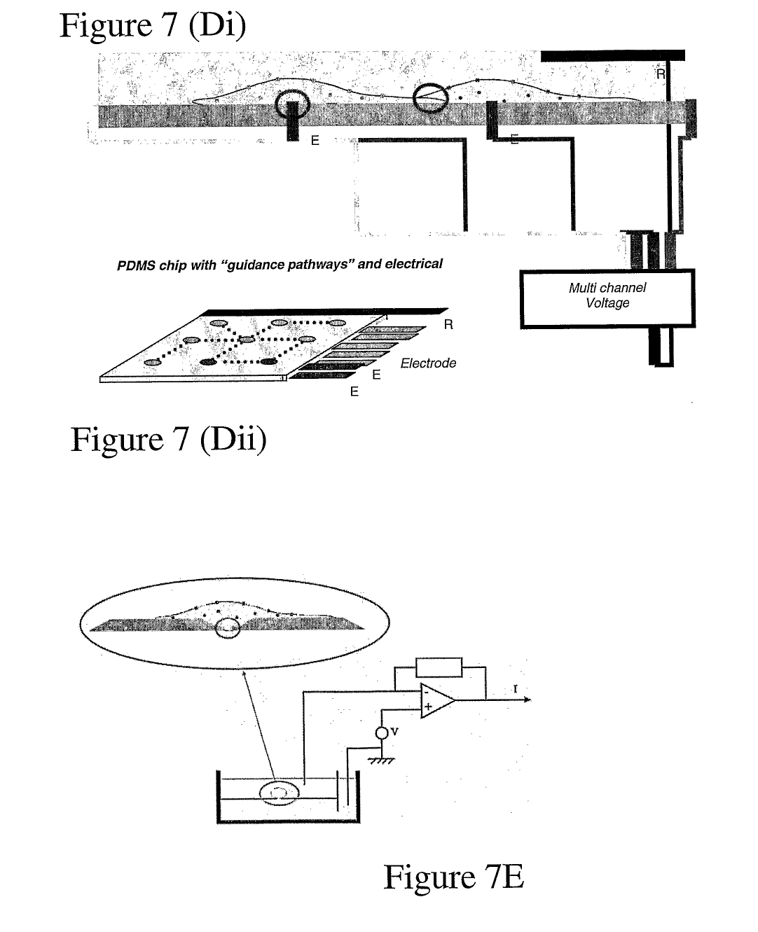

FIG. 7 is a schematic description of an embodiment of a substrate such as a chip for a patterned cell network such as a neural network (7a), an MEA interface electrode array (7b) a microfluidic array (7c), a synthetic neural network MEA interface (7di), a PDMS chip with guidance pathways and electrical contacts (7dii), and electrical contacts (7dii), and a patch-on-chip interface (7e).

In an embodiment of the invention there is provided the use of substrates and/or substrate/cell combinations disclosed herein in the examination of cellular responses to agents of interest. Agents of interest may include pharmaceuticals, pharmaceutical candidates, small molecules, oligopeptides, polypeptides and derivatives thereof, DNA's, RNA's carbohydrate-derived compounds, hormones and hormone derivatives, neurotransmitters and their derivatives, agonists and/or antagonists of cell surface or internal cellular receptors or proteins of interest found in or on the cultured cells.

In an embodiment of the invention there is provided a cell-growth substrate, said substrate comprising:
an electrically insulating membrane supported by a solid wafer having microholes extending across it;
said solid wafer having apertures defined therein such that channels are provided across said support;
the channels in the inert substrate being located in substantial alignment with microholes in the membrane so as to provide a passage across both the membrane and the support;
a substantially rigid enclosing layer of substantially inert material sealably engaging portions of the substrate;
said enclosing layer having defined therein thicker and thinner regions such that, in combination with the enclosing layer, a series of channels extending substantially parallel to the membrane are defined;
cell guidance regions of substantially inert material secured to the exposed surface of the membrane such that at least some microholes with aligned channels remain open.

It will be appreciated that the membrane may be formed on the desired support or formed elsewhere and transferred onto the desired support. The general purpose of the membrane is to facilitate the fabrication of a precise microhole. Thus, any suitable structure for that purpose will suffice.

As used herein, a “cell-suitable membrane” is a membrane which is capable of supporting the growth of adherent cells of at least one type. In some instances the membrane will be a thin film such as silicon nitride (SiN) or a heavily boron doped layer of an Si substrate, polyimide, etc.

As used herein, a “cell-suitable membrane” is a membrane which is capable of supporting the growth of adherent cells of at least one type. In many instances, coating the cell contact portion of the membrane with a combination of a parylene thin film and polylysine treatment will make it cell-suitable even if it was not cell-suitable prior to coating.

It will be understood that microholes may vary in size depending on the cell type of interest. In some instances, microholes in a given membrane will preferably have a diameter of between about 0.5 to 10 μm. In some instances a microhole diameter of between about 1 and 7 μm will be desired. In some instances, a microhole diameter of between about 2 and 6 μm will be desired.

The enclosing layer may be produced from any one or combination of materials which is substantially inert to the medium and conditions intended for use with the cell system to be studied. The enclosing layer is preferably made from a material which is substantially rigid or insufficiently elastomeric to collapse during the intended use. In some instances a curable polymer will be desired. In other instances a material which must be formed into the desired shape by machining, chemical etching, or another process whereby a portion of an original whole is removed, will be preferred.

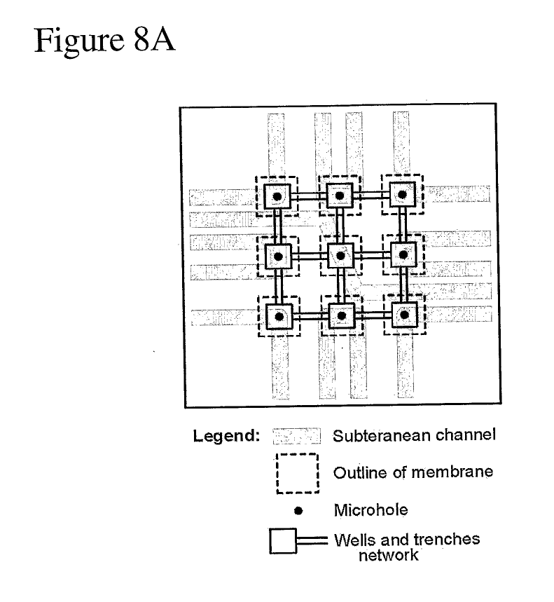

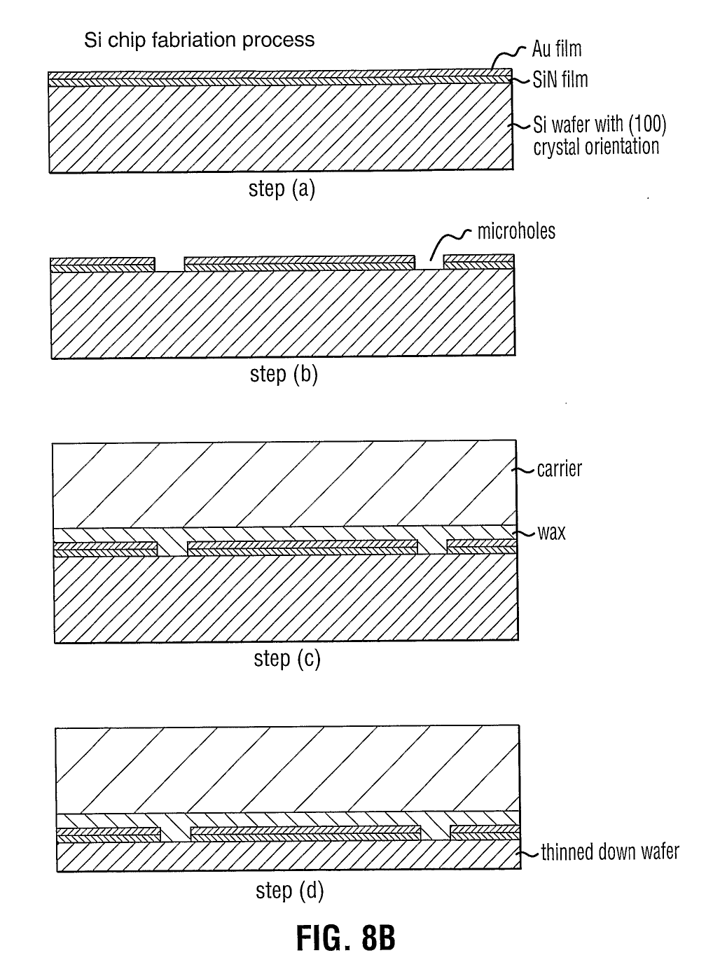

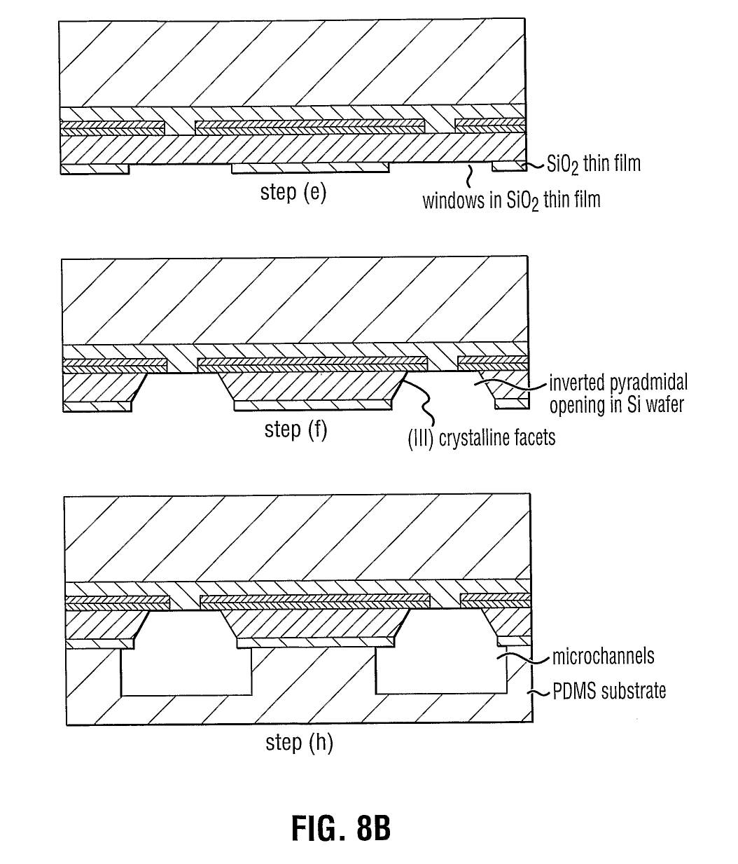

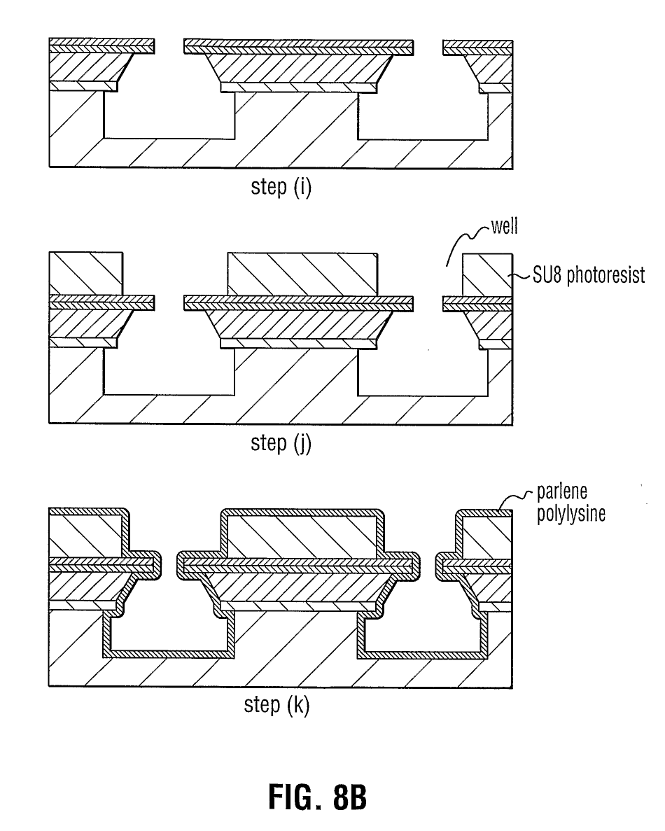

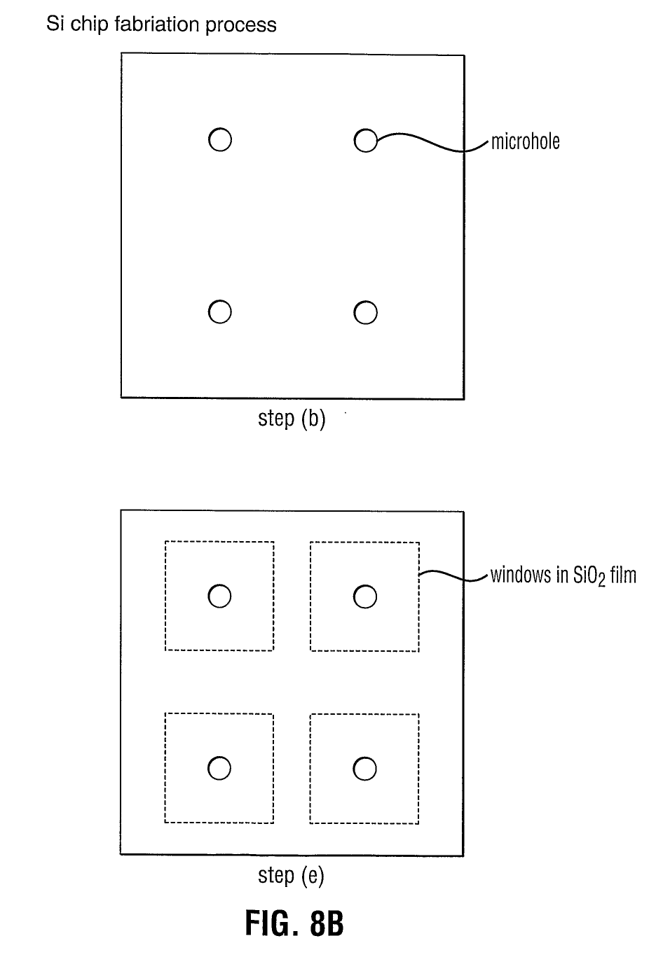

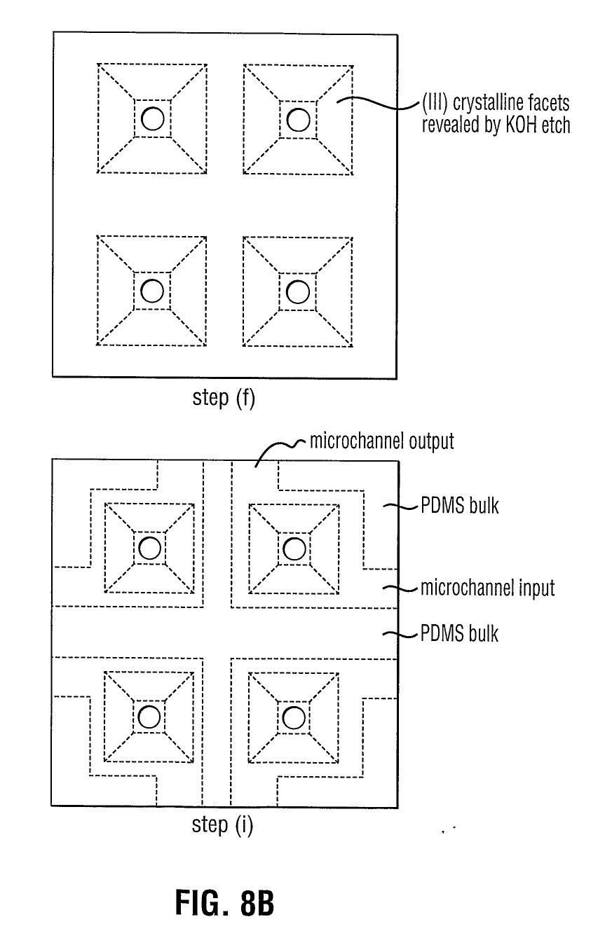

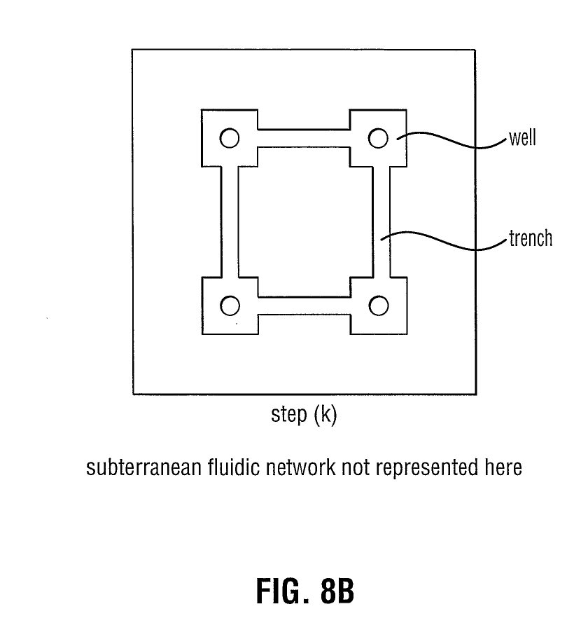

The guidance regions may be made from any single or combination of materials which is substantially inert in the culture medium and conditions intended for use with the cell type of interest. In some instances the guidance regions provide a less favorable surface for adhesion by that cell type than is provided by the membrane. Guidance regions of this type are called “patterned growth guidance regions.” In some instances guidance regions will be formed as “wells” and “trenches” on the membranes. An example of an embodiment of this is in FIG. 8.

In some instances it will be desired to maintain the ratio of average cell volume of the cell type for examination to the channel volume. In some cases the range of ratios (cell volume:channel volume) will be between about 1×10⁻¹⁹ liters (cell): 0.2 mm³ (channel) and about 1×10⁻¹⁴ liters (cell): 0.001 mm³ (channel). In some cases the range of ratios (cell volume: channel volume) will be between about 1×10⁻¹⁸ liters (cell): 0.1 mm³ (channel) and about 1×10⁻¹⁵ liters (cell): 0.01 mm³ (channel). These ranges are provided by way of example only and it will be appreciated that a wide range of ratios are possible, depending on the cells, conditions and particular substrate configurations employed and the objects and duration of the study to be conducted.

FIG. 8A depicts a schematic representations of an embodiment of substrates for use with a cell network such as a neural cell network. Wells and trenches are conducive to selected implantation of neurons and directed growth of neurites (FIG. 8A(i) is of a pattern with 20 μm square wells, 3 μm wide trenches, all being 70 μm deep). Micro-hole membranes have been described in the literature as allowing monitoring the electrophysiological activity of ion channels in neurons. Subterranean microfluidics channel allow both recording this activity and delivery of drugs or other compounds of interest to the cell to allow chemical patch-clamping and other studies.

In FIG. 8A the top layer (solid lines): cell placement and directed growth (wells and trenches network). Micro-holes (black dots): join top to subterranean network. In this embodiment the holes are very small (3-5 um) and are formed in a membrane (dashed lines). The bottom layer (grey): microfluidic channels that connect to each well separately.

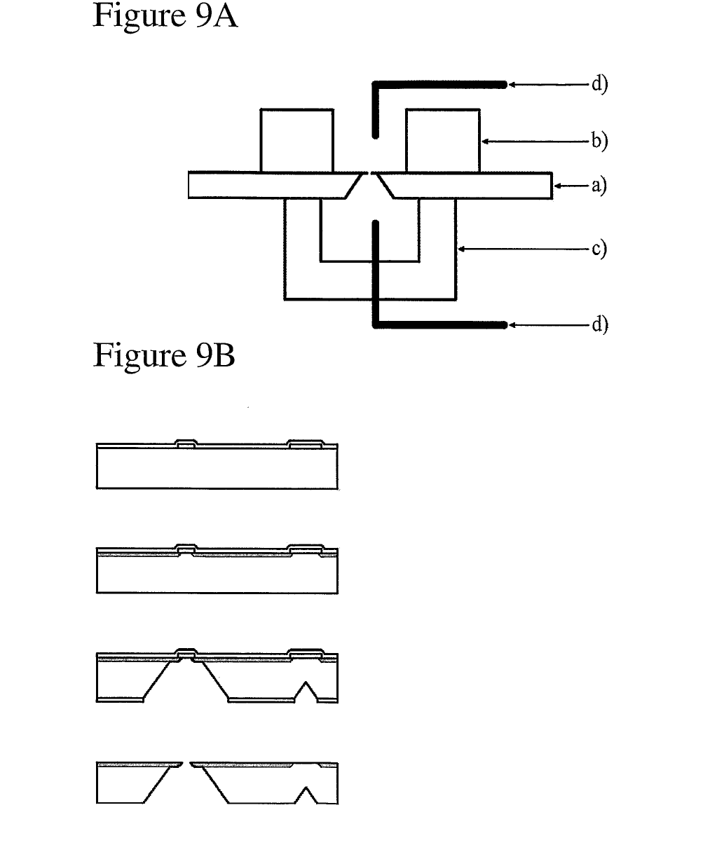

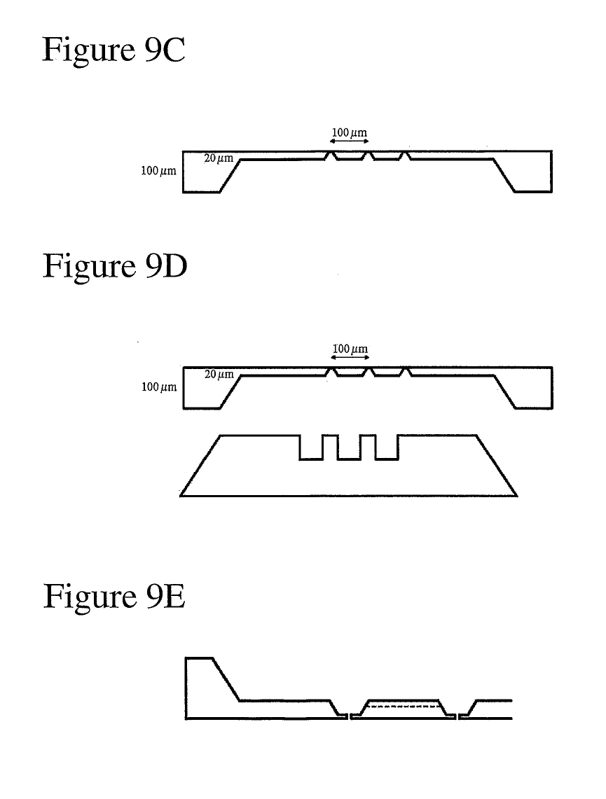

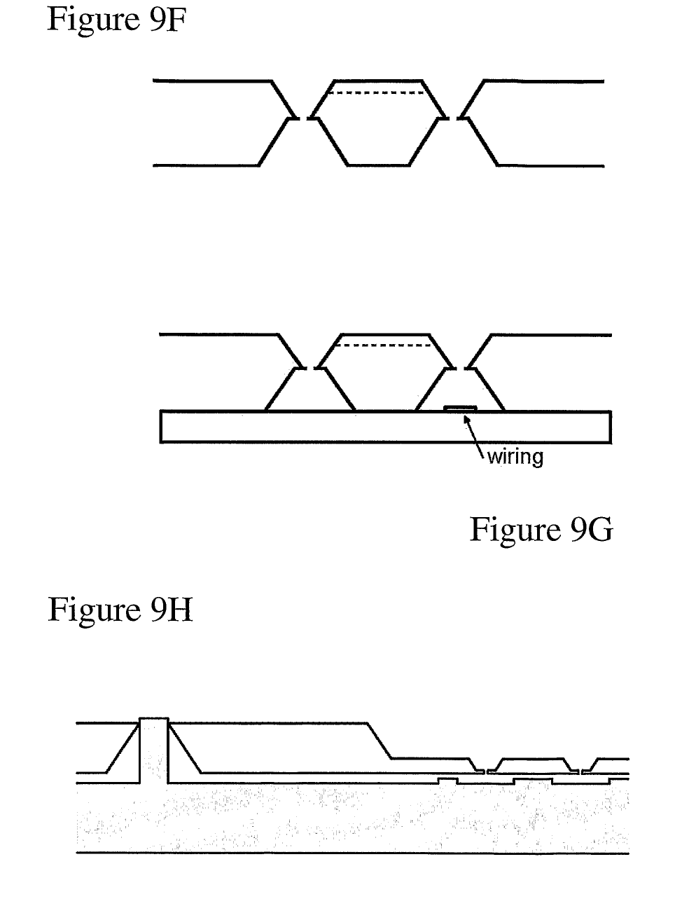

FIG. 9(a) is a side-view of an embodiment of the membrane micro-hole and explains how cell activity is monitored. It contains 4 parts (all within FIG. 9(a); a) a substrate carrying a membrane with micro-hole, b) cell container, c) microfluidic channels, and d) electric connections.

### EXAMPLE 2

Fabrication

It will be appreciated that different fabrication methods are possible in light of the disclosure herein. By way of non-limiting example, two different approaches (one using Si (2.1), the other using PDMS (2.2)) are described.

2.1) First Fabrication Method: Si Wafer Based Neurochip

2.1.1 describes the general method of fabrication and an actual recipe. Paragraphs 2.1.2 to 2.1.8 describe possible variations to the process, and their advantages.

2.1.1) General Description of an Example of a Fabrication Method

In an embodiment of the invention there is provided a method of producing a chip suitable for use in growing cells so as to promote growth of structured two-dimensional networks, said method described in FIG. 8B, comprising:
a) obtaining a SiN/Au thin film on a Si wafer with a (100) crystalline orientation;
b) creating microholes in the SiN/Au thin film (in some instances preferably having a diameter of between about 1 μm and about 5 μm) (in some instances a single hole may be desired, in other instances a sieve structure may be desired);
c) bonding the front of the wafer to a carrier with wax or another sacrificial layer (that can later be released);
d) thinning down the wafer by lapping to preferably a thickness of between about 25 and 75 μm;
e) obtain a SiO₂ thin film mask in the back of the wafer and create (preferably about 75-125 μm) more preferably about 100 μm windows in the SiO₂ thin film mask, said windows being aligned so as to connect to a microhole;
f) etch the Si wafer in a KOH solution through the windows in the SiO₂ mask that will reveal facets in the Si crystals, thereby creating an inverted pyramid structure resulting in an approximately 50 μm square SiN/Au membrane including the micro-hole;
g) obtaining a Poly-Dimethyl Siloxane (PDMS, also known as Silicone) chip defining channels with a 200 μm pitch;
h) bonding the PDMS chip to the Si chip such that a channel is positioned over a microhole in substantially sealing engagement;
i) releasing the Si chip from the carrier by melting, dissolving or otherwise removing the sacrificial layer that bonds them;
j) defining a network of wells and trenches in alignment with micro-holes such that each micro-hole be centered at the bottom of a well and connected to other wells via trenches (For example a SU8 photoresist can be spun on the top of the Si chip, aligned with a lithography mask, exposed to UV light, developed and then also coated with a thin film of parylene;
k) coating the entire chip with parylene, a bio-compatible, electrically insulating plastic that needs to be applied in a thin enough film so as not to plug the micro-hole (preferably less than 1 μm), and polylysine or other thin-film known to promote the implantation of different types of cells.

As used herein, the term “tower” refers to a solid structure which, when used as a mold, results in a well in the resulting product. In some instances a tower may have a square cross-section. However, it will be apparent that other shapes are possible including rectangular, circular, elliptical, and irregular shapes.

As used herein, the term “wall” refers to a solid structure which, when used as a mold, will result in the connection of two wells in the resulting product. In some instances a wall will have a narrow rectangular cross-sectional area. However, it will be understood that other shapes are possible. Well size and shape may be selected based on the cells and conditions for study and, in light of the disclosure herein, it will be within the ability of one skilled in the art to do so.

It should be understood that substantial variations can be brought to the process without affecting the spirit of the invention. In step a), Au is thought to be advantageous as a mask for etching SiN and as a protection against damage in subsequent steps, but it's use is optional and it could advantageously be replaced by Ni, Al, Cr and many other metals used in semiconductor technology; its nature is not critical to the application since the chip can be passivated with a bio-compatible plastic film in step g). The SiN layer itself can be replaced by silicon dioxide (SiO₂), a metal film, or polymers such as polyimide, as long as the material of the substrate can be etched selectively to it. The Si wafer itself can be replaced by other types of substrates, regardless of their electronic properties or bio-compatibility (glass, metal foils), but Si is thought to be particularly advantageous as fabrication processes are well-known to the semiconductor industry with that material. The SiO₂ thin film mask in step e) can be replaced by other material known to be selective to Si in a KOH solution: for example SiN itself. The KOH etching step may be replaced by other solutions known to etch Silicon, such as hydrazine, tetramethylammonium hydroxide or other solutions known to the state of the art (see Thin Film Processes, J. L. Vossen and W. Kern, Academic Press, NY), or by dry etching techniques such as reactive ion etching employing sulfur hexafluoride (SF6) as the reactive gas (same ref). For all those techniques, a different thin film mask as defined in step e) will be appropriate. PDMS in step g) can be replaced by any curable polymer, such as the UV-curable epoxy 1191-M provided by Dymax Corp. and commonly used as a medical device adhesive, or any rigid substrate like Si or glass: PDMS is thought to be advantageous as it is flexible and will bond easily with the rigid Si substrate. SU8 in step j) is thought to be advantageous as tall structures capable of effectively guiding the implantation of neurons and growth of neurites can be obtained in a single lithographic step, but it may be replaced by a film etched by methods similar as in step e).

FIG. 8B depicts an embodiment of this method with reference to the lettered steps.

In an embodiment of the invention, the membrane in which microholes are formed is produced by imaging a lithography mask on the membrane. In some instances it will be desired to put designs on the membrane.

In light of the disclosure herein, it is within the capacity of one skilled in the art to produce different membrane designs. This approach can be used to produce sieve-like structures instead of single microholes. It will generally be desired to produce sieves having about the same cross-sectional area as would a conventional microhole in the same circumstances. However, in some instances, larger sieve structures could be desired and produced.

TABLE 1
Recipe for the embodiment of the fabrication
process outlined in FIG. 8B

| | Step |
|---|---|
| a1 | SiN Plasma Enhanced Chemical Vapor Deposition, 1 um. |
| a2 | Ti/Au (100/3000A) e-beam evaporation |
| b1 | Litho 1, defining 3 um holes. |
| b2 | Wet etch in KI solution + few seconds in HF. |
| b3 | RIE etch of SiN. Strip photoresist of Litho 1. |
| c | Wafer bonding on carrier with wax. |
| d | Wafer lapping, down to 50 um. Polish. |
| e1 | Backside growth of 5 um SiO2. |
| e2 | Litho 2, using back to front alignment, defining 100 um windows on |
| | backside that will result in 50 um openings under top SiN |
| | membrane. |
| f | Si etch, KOH, 80 C. |
| g1 | On separate Si wafer: |
| | Litho 3 using SU8-50 for 100 um wide channels with 200 um pitch. |
| g2 | Replicate channels in PDMS to obtain self-standing PDMS film |
| | (1 mm thick) with channels on top. |
| | Bond PDMS film to glass substrate. |
| h | Spin PDMS thin film (5-10 um) on PDMS chip |
| | Align to backside of membrane wafer, cure PDMS |
| i | Remove carrier by heating wax. |
| j | Litho 4 in SU8-50 on front side of Si membrane to define wells and |
| | trenches that guide cells. |
| k | Parylene evaporation, 1 um, for electrical passivation. |
| | Immerse chip in polylisine solution |

2.1.2) SiN Membrane being Replaced by Highly Boron-Doped Si Layer

Instead of using a SiN membrane one may use boron doped Si (Si:B) as a KOH etch stop. A Si membrane can have better physical properties than SiN. It is a strong, single crystal material and is a perfect match with the substrate. This process also avoids the use of PECVD film growth and ICP etching. See FIG. 9(b) and Table II for a schematic description.

A specific fabrication process can be given by replacing the first two steps in section 2.1.1 General description of fabrication method with the following two steps.

a) Pattern a thermally grown SiO₂ film on a (100) Si substrate to mask boron diffusion at locations of microholes.

b) Using a high temperature anneal, diffuse boron into the top Si surface using a suitable boron source such as a spun on boron silicate film. This boron doped layer will act as an etch stop for the KOH etch, creating a membrane. The microholes will be formed during the KOH etch, since boron was masked from these areas.

Steps c) to k) could be the same as described in the general process description, section 2.1.1.

TABLE II

| | Step |
|---|---|
| 1 | Starting (100) Si wafer with a thermal oxide to mask boron diffusion |
| 2 | The oxide is patterned to mask boron diffusion at the orifice as well |
| | as define allinment marks |
| 3 | A boron doped silicate film is spun onto the top of the wafer |
| 4 | A high temperature anneal diffuses the B about 2 μm into the wafer, |
| | leaving an opening under the mask |
| 5 | A SiN mask is patterned on the back of the wafer and the Si is |
| | etched with KOH from the back |
| 6 | The masking SiN and the boron doped oxide layer are removed |

2.1.3) Thin Si Substrate

The anisotropic etching of Si results in pits with walls sloped at an angle of 54.74°. This limits the spacing of pits. In order to have small enough pit spacing, in some cases the starting Si wafer may have to be less than 50 μm thick. One method of doing this is to bond the Si wafer to another substrate and thin by mechanical or chemical means. An alternative would be to thin millimeter sized areas and leave the bulk of the wafer thick enough to be mechanically self supporting. Anisotropic etching could be used to thin these selected areas. One issue with this is that one may have to pattern the bottom of the etch pit. This can be done by projection lithography (or by electron beam lithography). See FIG. 9(c).

2.1.4)—Alignment of Membrane and Microfluids Parts

The general process description, section 2.1.1, step h) involves bonding two layers. These layers could be aligned optically, but the sloped (111) surfaces formed in the Si wafer during anisotropic etching could be used to allow mechanical alignment. In this case, the PDMS fluid channel layer would be shaped to exactly match features in the Si wafer, which will guide the positioning as the two pieces are brought together. See FIG. 9(d).

2.1.5)—Wiring on Wafer Front Side

This approach provides an alternative “up-side-down” version of the membrane. In this approach, one can fabricate layers of metal and insulator on the polished (now bottom) side of the wafer. A microfluidics part is bonded onto that. There are pits and grooves on the top side to contain the neurons and guide the arms. A SU-8 or similar layer is patterned on top in cases where it is necessary or desirable to confine the cells.

2.1.6)—Combine Membranes, Neuron Pit, and Fluidics

Here the three parts (membrane with microhole, cell container, and microfluidic channels) are all micromachined in a Si wafer. Also, as shown by the dashed line, it is possible to make trenches in the top surface to connect the cell pits.

In one embodiment processing of the above can be achieved by:

1) etch from back. 2) oxidize and pattern oxide for boron doping. This would preferably be accomplished by projection lithography (or electron beam lithography). Alternatively, a SiN layer could be grown and used as the membrane. 3) etch from the top. A single etch step can be used to form pits plus connecting trenches. For example, see FIG. 9(f). A possible layout of the micro-fluidic channels is sketched in FIG. 9(h). The fluid channels would be completed by bonding a flat sheet of suitable material to the bottom of the Si wafer. This bottom layer could include wiring for electrical connections. See FIG. 9(g).

In some instances it will be desirable to coat the assembled substrate in a substantially non-conductive coating, such as parylene.

2.1.7)—Alignment Peas and Slots

Alignment slots can be etched completely through the Si wafer, which, if desired, can match with pegs in a PDMS section to act as an alignment guide during bonding. See, for example, FIG. 9h.

2.1.8)—Polyimide Membrane

In some instances it may be desirable to use polyimide to replace the more expensive and more complex membrane processes involving SiN or Si:B. Using polyimide will still allow the formation of an accurate micro-hole. Polyimide is tough and dimensionally quite stable. When fully cured it is resistant to most solvents and acids. It is also stable at temperatures up to 400° C.

The following modifications to the general process description, section 2.1.1, provide a possible process using a polyimide membrane.

a) obtaining a fully cured polyimide layer on a (100) Si wafer. The thickness of the polyimide could be, for example, 2 μm. This would be coated with a metal film, which would be patterned to define the microholes. The metal could be Ti, Ni, Au, Cr, or others.

b) Etching microholes in the polyimide using a suitable method, for example an oxygen plasma etch.

The general steps c) to e) of section 2.1.1 could remain the same. Step f) would change as follows:

f) etch the Si wafer in a KOH solution through the windows in the SiO2 mask that will reveal (111) facets in the Si crystals, thereby creating an inverted pyramid structure. The KOH etch is preferably stopped with a thin layer of Si remaining before reaching the polyimide layer. This is because the KOH will etch the polyimide. The etching process can be completed with a short isotropic etch to remove the final Si and exposing the polyimide membrane. A suitable isotropic etchant would be a mixture of hydrofluoricacid, nitric acid, and acetic acid. Steps from g) to k) could remain the same.

FIG. 8 depicts an embodiment of a basic 8-orifice chip design with flow-through channels.

2.2) Second Fabrication Method: PDMS Based Neurochip

2.2.1 describes the general method of fabrication and an actual recipe. Paragraph 2.2.2 describes possible variations to the process using existing AFM tips.

2.2.1) General Description of Fabrication Method

There is disclosed a technique that adapts molding to an aligner so as to allow 3D features formed on wafers by conventional micromachining (and in some cases by replication) to be assembled and contacted, and the space in between them to be filled with a curable polymer. The method has been demonstrated with epoxy glues and PDMS as the materials of the final 3D mold; however, in light of the disclosure herein, one skilled in the art could readily see alternatives which also form part of the invention.
a) obtaining a SiN thin film mask on the surface of a Si wafer with a (100) crystalline orientation and create (preferably about 75-125 μm) more preferably about 100 μm square windows in the thin film mask, with a 200 um pitch;
b) etching the Si wafer in a KOH solution to reveal (111) crystalline facets creating an inverted pyramid;
c) forming the complementary feature of a pyramid by applying a thick polymer, on top of the Si wafer and cross-linking or curing it, then peeling it off the Si wafer;
d) obtaining a tower-and-wall pattern with a 200 μm pitch on a second substrate, for example a SU8 photoresist on a Si wafer patterned by conventional optical lithography;
e) optionally applying anti-stick treatments to the two patterned substrates, aligning the tower-and-wall pattern to the pyramid pattern and filling the space in between with a curable polymer—the contact area between the apex of the pyramids and the towers forming, in complementary moulding, a microhole, the size of which can be controlled by the elasticity of the materials employed and the pressure applied;
f) curing the polymer and removing the two substrates;
g) obtaining a PDMS chip defining channels with a 200 μm pitch;
h) bonding the PDMS chip to the Si chip such that a channel is positioned over a microhole in substantially sealing engagement;
i) optionally coating the entire chip with parylene and/or polylysine or other thin-film known to promote the implantation of different types of cells.

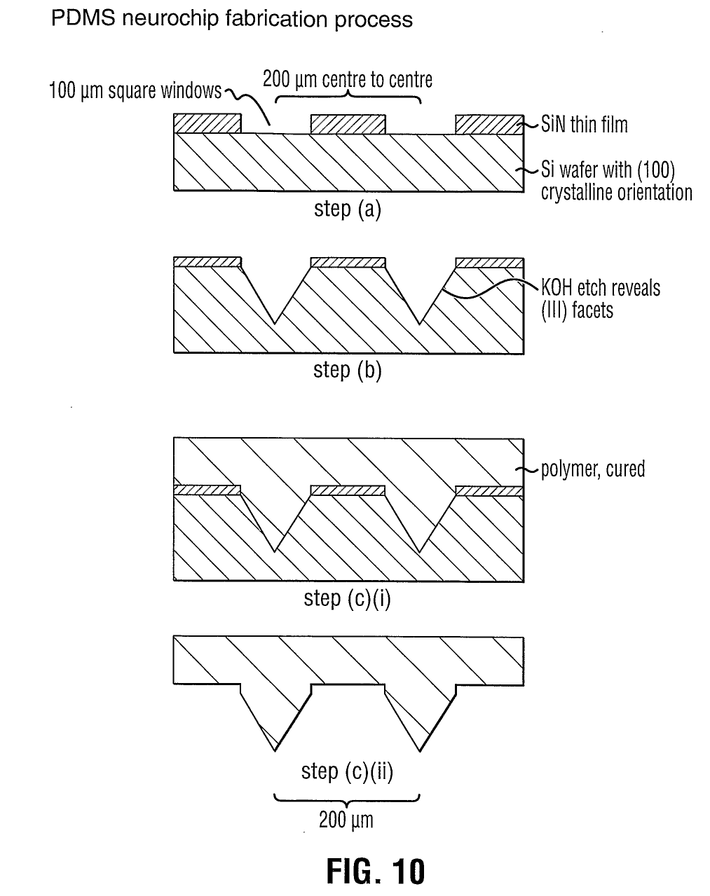

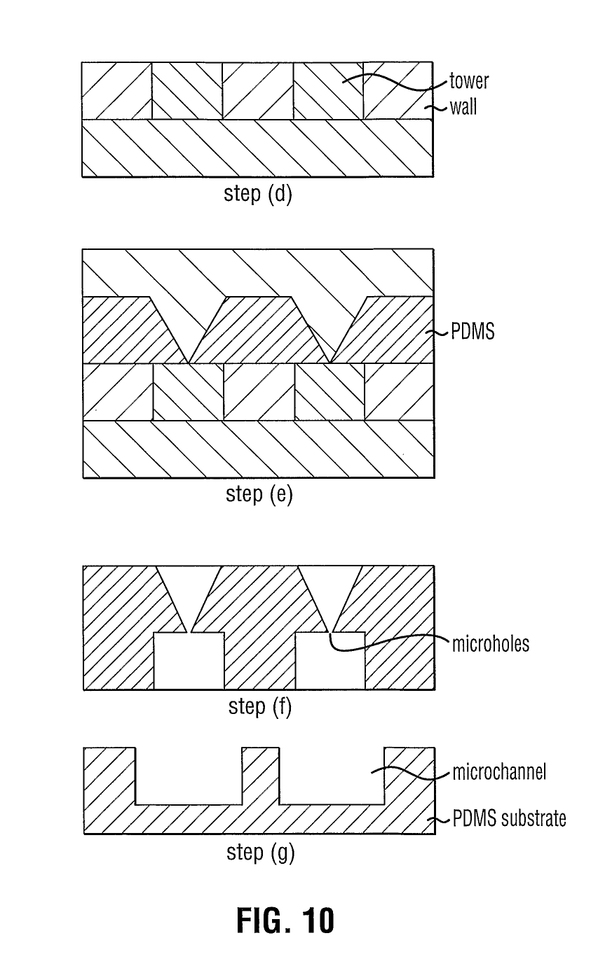

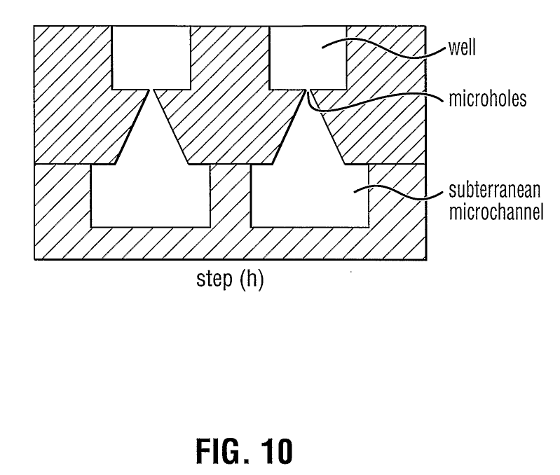

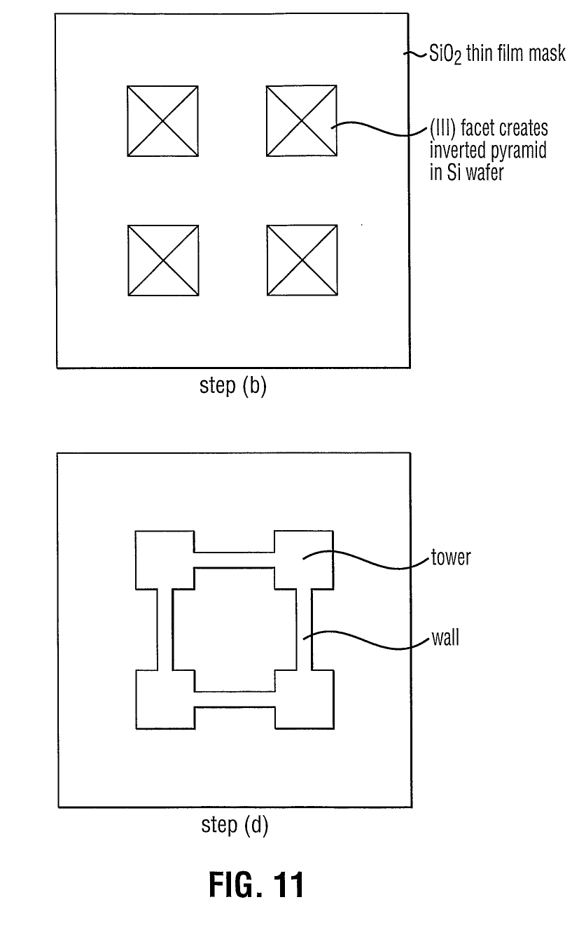

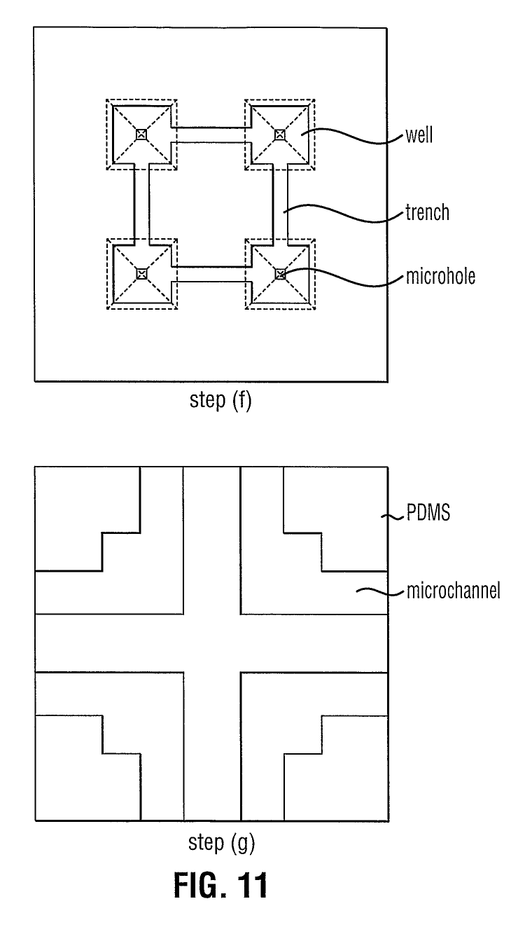

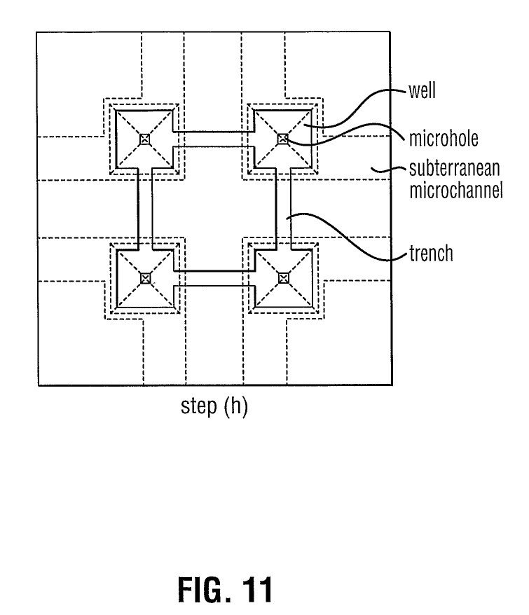

FIGS. 10 and 11 and related Table III set out steps for an embodiment of the production and fusing of two PDMS chips.

TABLE III

| | Steps |
|---|---|
| a1 | SiN Plasma Enhanced Chemical Vapor Deposition, 1 μm. |
| a2 | Litho 1, defining 100 square openings |
| b | Si etch, KOH, 80 C. |
| c | Replicate channels in PDMS to obtain self-standing PDMS film (1 |
| | mm thick) with pyramids on top. Bond PDMS film to glass substrate. |
| d | Litho 2 in SU8-50 on Si wafer to define towers and walls. |
| e1 | Align Si wafer with replicate obtained in C, and contact with |
| | controlled force so apex of pyramids are flattened on SU8-50 to |
| | 3-5 μm squares |
| e2 | Fill space with PDMS; |
| f | Cure; peel Si wafer and PDMS replicate obtained in C |
| g1 | Litho 3 in SU8-50 on second Si wafer to walls as complement of |
| | channels. |
| g2 | Replicate channels in PDMS to obtain self-standing PDMS film |
| | (1 mm thick) with channels on top. |
| h | Spin PDMS thin film (5-10 μm) on PDMS replicate obtained in |
| | step G Align replicate obtained in G with replicate obtained in F; |
| | cure PDMS. |

2.2.2) Variation Using an AFM Tip or Equivalent Structure for the Formation of the Micro-Hole and the Microfluidic Channels

In an embodiment of the invention, a variant of the process described in FIGS. 10 and 11 is employed. Steps (a) to (c) and (g) are omitted and a tip connected to a beam is employed to form the microhole (the apex of the tip in contact with a tower defines the microhole) and the beam typically defines an open channel to be closed in an assembly step, which will typically not require alignment. The tip and beam may be formed from any suitable material. A material will be suitable if it is sufficiently firm to define the microhole and channel and can be removed once these structures have been formed.

It will be understood that the “beam” may be contoured and/or bendable to permit formation of channels in various directions and/or dimensions.

AFM stands for Atomic Force Microscope. This example relates to an AFM tip because such tips are readily available. However, it will be understood that any suitably-sized tip having an extension thereof will suffice, provided that the tip dimensions is suitable to form the desired size of microhole and the extension is of a suitable size and shape to define the desired channel.

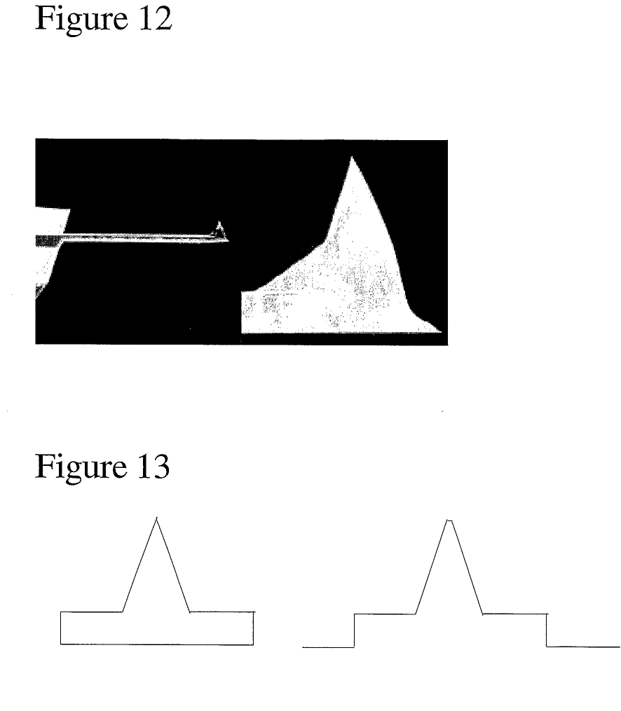

An AFM tip is composed of a tall sharp tip, usually in Si, at the end of a cantilever. This general shape can be used to form a membrane micro-hole using the 3D PDMS molding process disclosed herein, by which complementary shapes to the masters are formed. The apex of the tip, in contact with a pattern conducive to the placement of cells, forms the micro-hole; the cantilever forms a subterranean microfluidic channel. AFM tips fabrication processes are now in an industrial phase), but many different processes can be derived from Field-Emission Displays fabrication processes (see proceedings of International Vacuum Microelectronic Conference in JVST B, for example JVST B 15(2) (1997)). A picture of a type of AFM tip is shown in FIG. 12. The tip is 15 um tall, its apex has a radius of curvature under 15 nm; it is mounted on a 7 um thick, 33 um wide and 200 um long cantilever.

In order to make micro-holes, it is possible (though not necessary) to relax two parameters of the typical fabrication of the AFM tip: 1) the apex of the tip need only have a radius of curvature of a few microns and 2) the cantilever is not self standing but etched as a wall supported by the bulk of the Si wafer (see FIG. 13 comparing a standard AFM tip with the one preferred in this case).

In some instances the tip will preferably be taller (50 μm); since its base rests on the cantilever-wall, that will in such cases preferably be wider (100 μm) and it would benefit from being taller for a better flow of the fluids (20 μm). All these changes are possible without changing the fabrication process.

The tip is preferably coated with an anti-sticking layer such as Teflon or Ti/Au, the thickness of which would be controlled such that the apex of the tip would preferably be no more than 5 μm, when 3 μm holes are desired. (coating thickness and tip size and shape can readily be adapted to a desired application, in light of the disclosure herein).

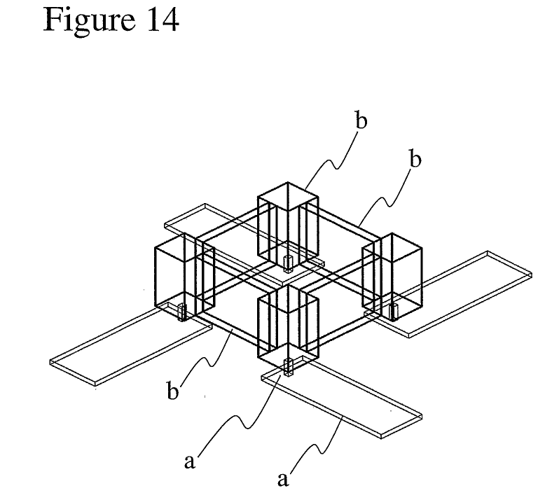

One or several cantilever tips is aligned to a pattern of towers and walls patterns (ie, the complement of wells and trenches) in SU8-50 on a glass or flexible plastic sheet, such that the tip is centered on the top of the tower (see FIG. 14 where the tips and cantilevers are indicated by “a” and the towers and walls by “b”).

It will be understood that the channels are preferably not blind (FIG. 14 is provided merely to describe a possible arrangement using an AFM tip or equivalent). When the alignment is made, the Si chip is flooded with PDMS such that it immerses the AFM tip (alternatively, the alignment can be performed if the PDMS is poured first).

After the PDMS is cured, the bottom Si chip is removed first, and then the PDMS is fused to a second glass or PDMS sheet to close the microfluidics channel. Finally, the top glass or plastic sheet is removed. This simple process forms a micro-hole membrane at the apex of the tip, aligned with a wells and trenches network on top and connected to a microfluidic channel as the complement of the cantilever.

A commercial AFM tip can be broken from its cantilever and fused to a PDMS or glass sheet by spinning a PDMS film, laying the tip on it, and curing the PDMS. The resulting mount is covered by a film 3 μm thick to blunt the tip and reinforce it.

This approach allows the formation of a substrate without a membrane. The microhole can be formed during the molding process.

### REFERENCES

Inclusions of a reference or other work is neither an admission nor a suggestion that it is relevant to the patentability of anything disclosed herein.
Corey D P, Stevens F C. Science and technology of patch-recording electrodes. In Single Channel Recording. B Sakman and E Neher, editors. 1983 Plenum Verlag, New York 53-86.
Opsahl L R, Webb W W. Lipid glass adhesion in giga-sealed patch-clamped membranes. Biophysical J. 1994; 66:75-84
Hamill, O P, Marty A, Neher E, Sakmann B, Sigworth F J. Improved patch-clamp techniques for high-resolution current recording from cells and cell-free membrane patches. Pfluers Arch. 1981; 391, 85-100.
Fertig N, Blick R H, Behrends J C. Whole cell patch clamp recording performed on a planar glass chip. Biophys J. 2002 June; 82(6):3056-62.
Stett A, Burkhardt C, Weber U, van Stiphout P, Knott T. Cytocentering a novel technique enabling automated cell-by-cell patch clamping with the cytopatch chip. Receptors Channels. 2003; 9(1):59-66.
Fertig N, George M, Klau M, Meyer C, Tilke A, Sobotta C, Blick R H, Behrends J C. Microstructured apertures in planar glass substrates for ion channel research. Receptors Channels. 2003; 9(1):29-40.
Klemic K G, Klemic J F, Reed M A, Sigworth F J. Micromolded PDMS planar electrode allows patch clamp electrical recordings from cells. Biosens Bpioelectron. 2002 June; 17(6-7):597-604
Fertig N, Blick R H, Behrends J C. Whole cell patch clamp recording performed on a planar glass chip. Biophys J. 2002 June; 82(6):3056-62.
Stett A. Burkhardt C, Weber U, van Stiphout P, Knott T. Cytocentering a novel technique enabling automated cell-by-cell patch clamping with the cytopatch chip. Receptors Channels. 2003; 9(1):59-66.
Fertig N, George M, Klau M, Meyer C, Tilke A, Sobotta C, Blick R H, Behrends J C. Microstructured apertures in planar glass substrates for ion channel research. Receptors Channels. 2003; 9(1):29-40.
Klemic K G, Klemic J F, Reed M A, Sigworth F J. Micromolded PDMS planar electrode allows patch clamp electrical recordings from cells. Biosens Bioelectron. 2002 June; 17(6-7):597-604
Klemic, Klemic, Read & Sigworth (Sep. 2, 2004) planar patch clamp electrodes; US 2004/0168912 A1
Klemic et al. Planar Patch Clamp Electrodes; US 2002/0064841 A1
Robertson (Dec. 16, 2004) High thoughput screening method and apparatus for monitoring ion channels; US 2004/021145 A1
U.S. Pat. No. 6,682,649, Substrate and a method of determining and/or monitoring electrophysiological properties of ion channels, Peterson et al
Huang et al, Sensors and Actuators A 105 (2003) 31-39
Lin et al, Sensors and Actuators B 99 (2004) 174-185
Y. Li, T. Pfohl, J. Kim, M. Yasa, Z. Wen, M. Kim, C. Safinya, “Selective surface modification in silicon microfluidic channels for micromanipulation of biological macromolecules,” Biomed. Microdev. vol. 3, pp. 239, (2001) International Vacuum Microelectronic Conference in JVST B, for example JVST B 15(2) (1997)
Y. N. Xia, G. M. Whitesides, Angew. Chem. Int. Ed. 37, 551 (1998)

We claim:

1. A method of studying cell membrane related activities, said method comprising:
(a) obtaining a cell adhesion surface having discrete orifices therein in communication with attached channels;
(b) culturing cells on the cell adhesion surface so that at least some of the cells grow over at least one orifice, such that the portion of the cell membrane in contact with the outer perimeter of the orifice forms a seal with the cell adhesion surface in the area immediately surrounding the orifice; and
(c) measuring changes in conditions within a fluid located within the channels connected to the orifices.

2. The method of claim 1 wherein the cell adhesion surface includes guidance regions.

3. The method of claim 1 wherein the channels have an inlet and a separate outlet in addition to being attached to an orifice.

4. The method of claim 1 wherein the fluid located within a channel contains an agent of interest.

5. A substrate, said substrate comprising:
a microhole containing layer having microholes extending through it;
a guidance layer of substantially inert material sealably engaging portions of a first side of the microhole containing layer;
said guidance layer in combination with the microhole containing layer defining a series of troughs extending substantially parallel to the microhole containing layer surface, wherein the trough walls are formed at least in part by the guidance layer and the trough base is defined at least in part by a region of the microhole containing layer defining a microhole.

6. The substrate of claim 5 wherein the microhole containing layer comprises a membrane having a first side forming the first side of the microhole containing layer and a second side in sealed engagement with a backing; said backing having aperatures defined therein such that channels are provided across said backing, at least some channels being substantially aligned with a microhole so as to provide a passage across the membrane and the backing.

7. The substrate of claim 6 wherein the backing is a solid wafer.

8. The substrate of claim 5 wherein the guidance layer is substantially rigid.

9. The substrate of claim 5 wherein at least a portion of the microhole containing layer is substantially electrically insulating.

10. A method of producing a substrate suitable for use in attaching and/or growing cells so as to promote development of structured cell networks in two or more dimensions, said method comprising:
a) obtaining a film on a first side of a substantially inert backing;
b) creating microholes in the film;
c) bonding the second side of the backing to a carrier,
d) obtaining a mask in the first side of the backing and creating windows in the thin film mask, said windows being aligned so as to connect to a microhole;
f) etching the backing through the windows in the mask, to create an inverted pyramid structure resulting in a membrane including the micro-hole;
g) obtaining a second chip defining channels;
h) bonding the second chip to the backing such that a channel is positioned over a microhole in substantially sealing engagement;
i) releasing the backing from the carrier;
j) applying a patterned growth cell guidance region on the first side of the membrane in alignment with micro-holes such that a micro-hole is located at the bottom of a well and the well is connected to other wells via trenches;
k) coating the resulting product with a bio-compatible, electrically insulating plastic so as not to plug the micro-hole, and polylysine or another suitable thin-film to promote the implantation of different types of cells.

11. The method of claim 10 wherein the microholes have a diameter of between about 0.5 μm and about 10 μm.

12. The method of claim 10 wherein the film is a thin film.

13. The method of claim 12 wherein the film is SiN/Au.

14. The method of claim 10 wherein the backing is an Si wafer.

15. The method of claim 14 further including, after step c: step c1 of: thinning down the wafer by lapping to preferably a thickness of between about 25 and 75 μm.

16. The method of claim 10 wherein, within step d, the windows in the mask are between about 75 and 125 μm across.

17. The method of claim 10 wherein the patterned growth cell guidance region comprises comprising a network of wells and trenches formed by the application of a substantially inert material to the membrane such that he walls of the trenches and wells are formed at least in part by the material and the base of the trenches and wells are formed at least in part by the membrane.

18. A method of producing a substrate suitable for use in growing cells so as to promote growth of structured networks in two or more dimensions, said method comprising:
a) obtaining a film on a first side of a Si wafer with a crystalline orientation;
b) creating microholes in the SiN/Au thin film;
c) bonding the second side of the wafer to a carrier with wax or another sacrificial layer,
d) obtaining a mask in the back of the wafer and creating windows in the thin film mask, said windows being aligned so as to connect to a microhole;
f) etch the Si wafer through the windows in the mask, thereby creating an inverted pyramid structure resulting in a membrane including the micro-hole;
g) obtaining a second chip defining channels with a defined pitch;
h) bonding the second chip to the Si chip such that a channel is positioned over a microhole in substantially sealing engagement;
i) releasing the Si chip from the carrier;
j) defining a network of wells and trenches in alignment with micro-holes such that a plurality of micro-holes are located at the bottom of a well and connected to other wells via trenches;
k) coating the entire chip with a bio-compatible material so as not to plug the micro-hole, and polylysine or another suitable thin-film to promote the attachment, growth and/or guidance of different types of cells.

19. The method of claim 16 wherein in step k the biocompatible material is an electrically insulating plastic

20. A method of producing a substrate suitable for use in growing cells so as to promote growth of structured networks in two or more dimensions, said method comprising:
a) obtaining a tip connected to a beam;
b) obtaining a backing having a carrier bonded to a first surface, said backing defining towers and walls along a second surface;
c) positioning the tip such that apex of the tip in contact with the top of a tower on the backing and the beam extends to and edge of the backing;
d) filing the space between the tip and the backing with a material which is fluid when applied but can be converted to a solid form;
e) converting the material of step d into a solid form;
f) removing the tip and the backing from the cured material to reveal a well structure with microholes and channels therein
g) where the tip was positioned such that its removal results in openings to the outside air in regions formed by the tip or the beam, closing off such openings to form closed channels except at the end of the channels defined by the beam;
h) optionally, coating the resulting product with a bio-compatible, electrically insulating plastic so as not to plug the micro-hole, and polylysine or another suitable thin-film to promote the implantation of different types of cells.

21. A method of forming an interface between a biological membrane and a substrate, said method comprising:
a) obtaining a substrate of claim 5;
b) culturing cells on the microhole containing layer/guidance layer surface of the substrate;
c) creating a patch-clamp connection between the cell and the substrate at a microhole.

22. The method of claim 18 further including a step d of monitoring electrical fluctuations in the channel below the microhole.

23. A method of producing a system suitable for use in studying whole-cell electrical responses to a stimulus, said method comprising:
a. obtaining a substrate of claim 5;
b. culturing cells on the membrane/guidance layer surface of the substrate in a culture medium such that at least one cell grows over a microhole;
c. creating a patch-clamp connection between the membrane and the substrate at a microhole;
d. rupturing a portion of the membrane over the microhole.

24. The method of claim 20 further including a step e of monitoring electrical fluctuations in the channel below the microhole.

25. The method of claim 21 wherein the electrical fluctuations measured includes at least one of voltage, current, capacitance.

26. Use of the substrate of claim 5 to assay the response of a cell to a stimulus.

27. Use of the method of claim 18 to study ion channel activity or membrane potential.

28. The substrate of claim 5 wherein the microhole in the membrane is defined by a plurality of adjacent holes to form a sieve-type structure.

29. The substrate of claim 24 wherein the sieve-type structure has a diameter of between about 1 and 10 μm.

30. A substrate, said substrate comprising:
a microhole containing layer having microholes extending through it;
at least one channel in sealing engagement with a microhole at a first end and being openable to the environment at a second end.
